(19) United States
(12) Patent Application Publication
(10) Pub. No.: US 2024/0293281 A1
(43) Pub. Date: Sep. 5, 2024

(54) GLOVE FOR PREVENTING AND RELIEVING TENOSYNOVITIS

(71) Applicant: Shanghai University of Medicine & Health Sciences, Shanghai (CN)

(72) Inventors: Zhifang YANG, Shanghai (CN); Qian HU, Shanghai (CN); Qingxia WANG, Shanghai (CN); Yue HAN, Shanghai (CN)

(21) Appl. No.: 18/646,378

(22) Filed: Apr. 25, 2024

Related U.S. Application Data
(63) Continuation of application No. PCT/CN2023/103717, filed on Jun. 29, 2023.

(30) Foreign Application Priority Data
Jan. 6, 2023 (CN) 202310018261.5

(51) Int. Cl. A61H 15/02; A61H 15/00

(52) U.S. Cl. CPC A61H 15/02; A61H 15/0078; A61H 2015/0071; A61H 2201/0207; A61H 2201/165; A61H 2201/501; A61H 2201/5043; A61H 2205/065

(57) ABSTRACT

A glove for preventing and relieving tenosynovitis is provided. The glove includes a glove body, a hand warming assembly is arranged at a lower part of the glove body; a control assembly and mounting bases are arranged at an upper part of the glove body. Each of the mounting bases is provided with an insertion block and includes a clamping sleeve; an adsorption magnetic ring is arranged at a lower end of the insertion block. A sliding rod elastically slides at a lower part of the insertion block; a heating plate is arranged at a lower end of the sliding rod; massage beads and a vibrator are arranged at the heating plate. The lower part of the insertion block is slidably provided inside the clamping sleeve, an inner wall of the clamping sleeve is circumferentially provided with a limiting magnetic ring.

### CROSS-REFERENCE TO RELATED APPLICATIONS

This application is a continuation of PCT/CN2023/103717, filed on Jun. 29, 2023 and claims priority of Chinese Patent Application No. 202310018261.5, filed on Jan. 6, 2023, the entire contents of which are incorporated herein by reference.

### TECHNICAL FIELD

The disclosure belongs to the field of tenosynovitis gloves, and in particular to a glove for preventing and relieving tenosynovitis.

### BACKGROUND

Tendon sheath includes two layers, the middle cavity is called synovial cavity, and the function of tendon sheath is to protect tendon. Excessive friction between tendon and tendon sheath leads to inflammatory reaction at the joint, which is called tenosynovitis. Tenosynovitis is a common disease in middle-aged and elderly people, but now tenosynovitis caused by work nature and trauma is more and more widespread in young people. Tenosynovitis mostly occurs in fingers, wrists and elbows. Tenosynovitis in wrists often occurs in the radial styloid tendon sheath. Tenosynovitis is a common disease and is mostly caused by external reasons, including long-term flexor of tendon or trauma, long-term friction between tendon and tendon sheath, local chronic inflammation, tissue edema and congestion. The conventional treatment method mainly relies on hormones. According to the literature, the common physical therapy adopts the theory of traditional Chinese medicine. Massage on important acupoints with hot compress may dredge meridians, relieve pain and reduce swelling, which is conducive to promoting blood circulation. Needle-knife therapy is a minimally invasive surgery, which cuts the ligament, completely removes the friction between tendon and tendon sheath, restores blood circulation, relieves pain and fundamentally cures the disease but it does great harm to patients.

The existing glove for preventing and relieving tenosynovitis has a relatively simple function, mostly for fixing or heating fingers, and does not show apparent or rapid effect, and therefore the use experience of patients is affected, and it is difficult to relieve the pain of patients' palms.

### SUMMARY

The technical problem to be solved by the disclosure is to overcome the shortcomings of the prior art and provide a glove for preventing and relieving tenosynovitis.

In order to solve the technical problems, the basic idea of the technical scheme adopt by the disclosure is as follows.

A glove for preventing and relieving tenosynovitis includes a glove body, a hand warming assembly is arranged at a lower part of the glove body. A control assembly and multiple mounting bases are arranged at an upper part of the glove body. An insertion block is arranged on each of the mounting bases: an adsorption magnetic ring is arranged at a lower end of the insertion block. A sliding rod elastically slides at a lower part of the insertion block; a heating plate is arranged at a lower end of the sliding rod: multiple massage beads are uniformly arranged at a lower end of the heating plate; and a vibrator is arranged at an upper end of the heating plate: and the adsorption magnetic ring and the vibrator are both located at a periphery of the sliding rod, and the vibrator is located below the adsorption magnetic ring.

Each of the mounting bases includes a clamping sleeve, the lower part of the insertion block is slidably provided inside the clamping sleeve, and an inner wall of the clamping sleeve is circumferentially provided with a limiting magnetic ring. The adsorption magnetic ring and the limiting magnetic ring are both located at peripheries of the heating plate and the vibrator; the adsorption magnetic ring is located at an upper end of the limiting magnetic ring: an outer wall of the clamping sleeve circumferentially is provided with a fixing plate: and the clamping sleeve and the fixing plate are both located inside the glove body.

Optionally, the glove body sequentially includes a fluff layer, an insulation layer, a reinforcing layer and an antifouling layer from inside to outside. A surface of the antifouling layer is formed with a circular groove corresponding to the clamping sleeve, the circular groove penetrates through the antifouling layer and the reinforcing layer. The clamping sleeve is installed at a periphery of an inner wall of the circular groove; the fixing plate is located between the reinforcing layer and the insulation layer; and a lower end of the inner wall of the circular groove is formed with a movable groove, the movable groove penetrates through the insulation layer. The heating plate is located in the movable groove.

Optionally, the upper end of the limiting magnetic ring is formed with two slotted holes, the sliding rod is positioned between the two slotted holes, and an electric-contact piece is arranged inside each of the slotted holes.

Optionally, the adsorption magnetic ring is formed with two through holes in a vertical direction, and the through holes penetrate the adsorption magnetic ring: the lower end of the insertion block is provided with a contact pin corresponding to the electric-contact piece: the contact pin penetrates corresponding one of the through holes, and a lower part of the contact pin is located inside the electric-contact piece.

Optionally, an outer periphery of the insertion block is formed with a ring opening, the ring opening is located at an upper end of the clamping sleeve, and the lower end of the insertion block is formed with a notch; an upper end of an inner wall of the notch is formed with a chute: an upper part of the sliding rod penetrates through the notch; an extrusion plate is installed at the upper end of the sliding rod: the extrusion plate is slidably provided inside the chute; and a control assembly is arranged between an upper end of the extrusion plate and an upper end of an inner wall of the chute.

Optionally, a side of the glove body is formed with an opening, an upper surface of the glove body is provided with an upper adhesive strip, and a lower surface of the glove body is provided with a lower adhesive strap corresponding to the upper adhesive strip.

Optionally, the hand warming assembly includes a palm layer, a hook surface is arranged on the palm layer, heating wires each arranged in an S shape are arranged inside the palm layer, and a lower surface of the glove body is provided with a loop surface corresponding to the hook surface, and the hook surface is located below the loop surface.

Optionally, the control assembly includes a housing installed on the glove body, a display screen is installed on the upper surface of the housing, multiple buttons are movably provided on an upper part of the housing, the buttons are located at one side of the display screen, and braille protrusions are arranged on an upper parts of each of the buttons.

Optionally, the housing is internally provided with a storage battery, a leakage protector, a controller, a vibration module, a heating module, a socket and a charging socket. The storage battery is connected with the leakage protector and the controller, the leakage protector is connected with the charging socket and the socket, and the controller is connected with the leakage protector, the vibration module, the heating module and the socket.

Optionally, the controller is connected with a wireless signal transmitting module, a wireless signal receiving module and a voice module, and the wireless signal transmitting module, the wireless signal receiving module and the voice module are all located inside the housing.

After adopting the above technical solutions, compared with the prior art, the embodiment has the following beneficial effects. Of course, it is not necessary for any product in the present disclosure to achieve all the following advantages at the same time.

Through the mounting bases, it is convenient to accurately install the insertion block according to the pain position of patients with tenosynovitis. Through the cooperation of the adsorption magnetic ring and the limiting magnetic ring, it is convenient to quickly install or disassemble the insertion block. Through the cooperation of the vibrator and the massage beads, the patient's tendon sheath is massaged, so that the muscles of the patient's hand are relaxed; and at the same time, the heating plate is cooperated to accelerate the blood circulation of the patient's palm tendon sheath, so that the accumulated interstitial fluid quickly disperses, the lump in the affected part of tenosynovitis quickly subsides and the pain of the patient is relieved.

The specific embodiments of the present disclosure will be described in further detail with reference to the accompanying drawings.

### BRIEF DESCRIPTION OF THE DRAWINGS

The attached drawings in the following description are only some embodiments, and other drawings may be obtained according to these drawings without creative efforts for ordinary people in the field. In the attached drawings:

FIG. 1 is a schematic structural view of a upper surface of a glove according to an embodiment of the present disclosure.

FIG. 2 is a schematic structural view of a lower surface of the glove according to an embodiment of the present disclosure.

FIG. 3 is a schematic structural diagram of a control assembly according to an embodiment of the present disclosure.

FIG. 4 is a schematic structural diagram of a hand warming assembly according to an embodiment of the present disclosure.

FIG. 5 is a schematic structural view of a section according to an embodiment of the present disclosure.

FIG. 6 is a schematic structural view at A in FIG. 5.

It should be noted that these drawings and written descriptions are not intended to limit the conceptual scope of the disclosure in any way, but to explain the concept of the disclosure to those skilled in the art by referring to specific embodiments.

### DETAILED DESCRIPTION OF THE EMBODIMENTS

The present disclosure will now be described in further detail with reference to the attached drawings.

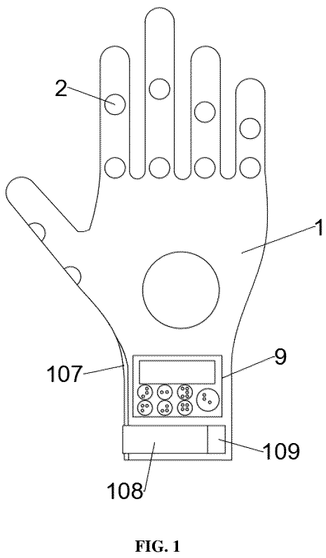

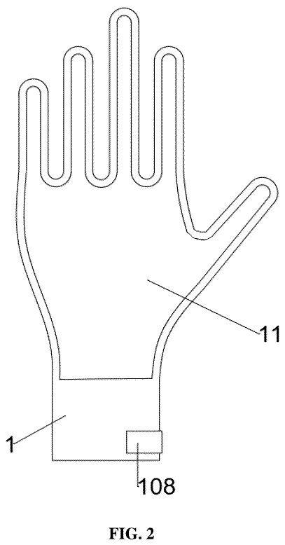

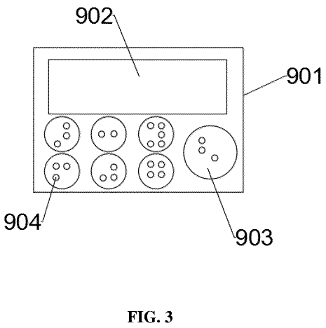

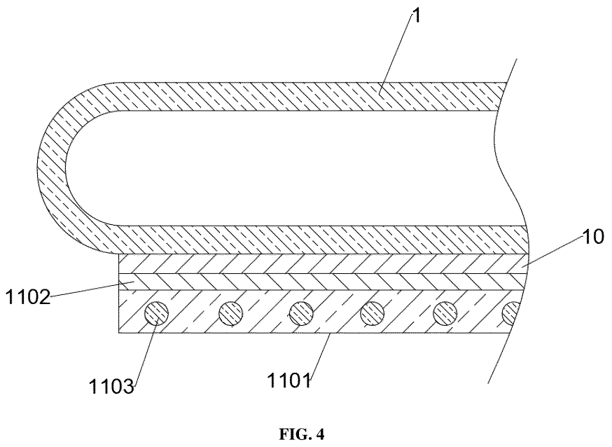

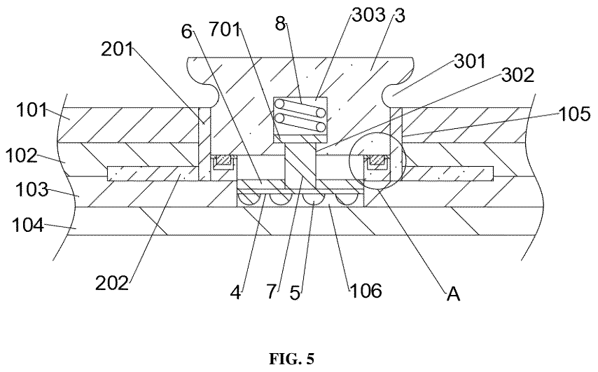

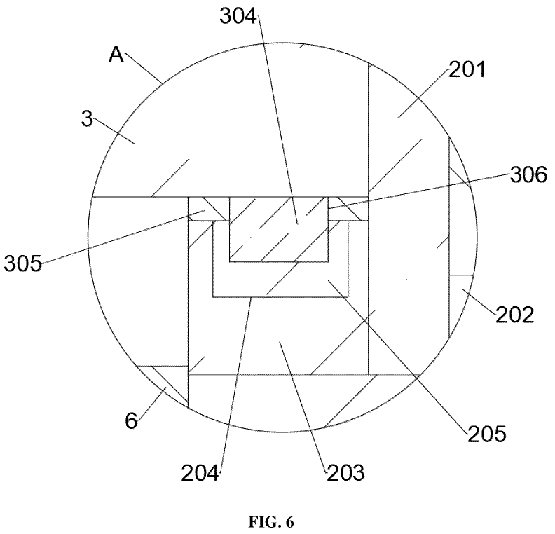

As shown in FIGS. 1 to 6, a glove for preventing and relieving tenosynovitis provided in this embodiment includes a glove body 1, a hand warming assembly 11 is arranged at the lower part of the glove body 1. A control assembly 9 and multiple mounting bases 2 are arranged at the upper part of the glove body 1, the mounting bases 2 are arranged corresponding to the positions of the flexor tendon sheath of the palm, the flexor pollicis longus tendon sheath and the stenotic tendon sheath of the styloid process of the radius. An insertion block 3 is arranged on each of mounting bases 2, and an adsorption magnetic ring 305 is arranged at the lower end of the insertion block 3. A sliding rod 7 elastically slides at a lower part of the insertion block 3, and the lower end of the sliding rod 7 is provided with a heating plate 4, and the lower end of the heating plate 4 is uniformly provided with multiple massage beads 5, and the upper end of the heating plate 4 is provided with a vibrator 6, and the adsorption magnetic ring 305 and the vibrator 6 are both located at the periphery of the sliding rod 7, and the vibrator 6 is located below the adsorption magnetic ring 305.

Each of mounting bases 2 includes a clamping sleeve 201, and the lower part of the insertion block 3 is slidably provided inside the clamping sleeve 201. A limiting magnetic ring 203 is circumferentially installed on the inner wall of the clamping sleeve 201. The adsorption magnetic ring 305 and the limiting magnetic ring 203 are located at the peripheries of the heating plate 4 and the vibrator 6, and the adsorption magnetic ring 305 is located at the upper end of the limiting magnetic ring 203. A fixing plate 202 is circumferentially installed on an outer wall of the clamping sleeve 201, and the clamping sleeve 201 and the fixing plate 202 are both located inside the glove body 1.

An application of one aspect of this embodiment is as follows: when in use, the patient inserts the insertion block 3 into the corresponding clamping sleeve 201 according to the pain position, so that the adsorption magnetic ring 305 is attracted to the limiting magnetic ring 203 for installation, and then the hand warming assembly 11 is stuck the lower surface of the glove body 1, and the patient puts the glove body 1 on the palm of his hand. And the control assembly 9 controls the heating plate 4 to work to generate heat to accelerate the blood circulation in the palm, and the vibrator 6 works to drive the heating plate 4 to vibrate. The vibration of the heating plate 4 drives the massage beads 5 to massage the patient's palm, so that the muscles of the patient's hand are relaxed. Meanwhile, the hand warming assembly 11 works to generate heat so as to warm the patient's palm, and heat and massage the patient's painful part, so that the accumulated interstitial fluid quickly disperses, and the lump at the affected part of tenosynovitis quickly subsides, and thus the pain of the patient is relieved.

The glove body 1 in this embodiment sequentially includes a fluff layer 104, an insulation layer 103, a reinforcing layer 102, and an antifouling layer 101 from inside to outside. A circular groove 105 corresponding to the clamping sleeve 201 is formed on the surface of the antifouling layer 101, and the circular groove 105 penetrates through the antifouling layer 101 and the reinforcing layer 102. The clamping sleeve 201 is circumferentially installed on the inner wall of the circular groove 105, and the fixing plate 202 is located between the reinforcing layer 102 and the insulation layer 103. The lower end of the inner wall of the circular groove 105 is formed with a movable groove 106, the movable groove 106 is used for moving of the heating plate 4, the massage beads 5, and the vibrator 6: the movable groove 106 penetrates through the insulation layer 103. The heating plate 4 is located in the movable groove 106. The comfort of wearing of patients is improved through the fluff layer 104, and the fixing plate 202 is installed between the reinforcing layer 102 and the insulation layer 103, so that the mounting bases 2 are tightly fastened.

The upper end of the limiting magnetic ring 203 in this embodiment is formed with two slotted holes 204, and the sliding rod 7 is located between the two slotted holes 204, and an electric-contact piece 205 is installed inside each of the slotted holes 204. The adsorption magnetic ring 305 is formed with two through holes 306 in a vertical direction, the through holes penetrate the adsorption magnetic ring 305. The lower end of the insertion block 3 is provided with a contact pin 304 corresponding to the electric-contact piece 205. The contact pin 304 penetrates corresponding one of the through holes 306, and the lower part of the contact pin 304 is located inside the electric-contact piece 205. When the contact pin 304 is misaligned with the electric-contact piece 205, the insertion block 3 rotates so as to drive the contact pin 304 to rotate. When the contact pin 304 is aligned with the electric-contact piece 205, the adsorption magnetic ring 305 and the limiting magnetic ring 203 are attracted together, so that the lower part of the contact pin 304 is clamped into the electric-contact piece 205, and the contact pin 304 is contacted through the electric-contact piece 205, so as to supply the heating plate 4 and the vibrator 6 with power for normal operation.

The periphery of the insertion block 3 in this embodiment is formed with a ring opening 301, the ring opening 301 is located at the upper end of the clamping sleeve 201, and the ring opening 301 facilitate the finger to be clamped to pull the insertion block 3. The lower end of the insertion block 3 is formed with a notch 302, and the upper end of the inner wall of the notch 302 is formed with a chute 303. The upper part of the sliding rod 7 penetrates through the notch 302, and the upper end of the sliding rod 7 is provided with an extrusion plate 701, the extrusion plate 701 is slidably provided inside the chute 303. A spring 8 is arranged between the upper end of the extrusion plate 701 and the upper end of the inner wall of the chute 303. When the vibrator 6 vibrates, the sliding rod 7 moves up to push the extrusion plate 701 to squeeze the spring 8, and then the spring 8 rebounds to push the extrusion plate 701 to move down.

The side of the glove body 1 in this embodiment is formed with an opening 107, an upper adhesive strip 109 is installed on the upper surface of the glove body 1, and a lower adhesive strap 108 corresponding to the upper adhesive strip 109 is installed on the lower surface of the glove body 1. After the glove body 1 is put on the palm of a patient's hand, the lower adhesive strap 108 is pulled to bypass the opening 107 and adhere to the upper adhesive strip 109, thereby tightening the wrist part of the glove body 1, thereby meeting the needs of different people.

The hand warming assembly 11 in this embodiment includes a palm layer 1101, the palm layer 1101 covers the finger part and palms part of the glove body 1. A hook surface 1102 is installed on the palm layer 1101, and heating wires 1103 each arranged in an “S” shape are installed in the palm layer 1101. The lower surface of the glove body 1 is provided with a loop surface 10 corresponding to the hook surface 1102, the hook surface 1102 is located below the loop surface 10. The loop surface 10 and the hook surface 1102 are the primary and secondary surfaces of a hook and loop fastener, so that it is convenient to attach the hand warming assembly 11 to the glove body 1. The heating wires 1103 are connected with an electric wire, and the electric wire is connected with a plug.

The control assembly 9 of this embodiment includes a housing 901 installed on the upper surface of the glove body 1, and a display screen 902 is installed on the upper side of the housing 901. Multiple buttons 903 are movably fitted on the upper part of the housing 901. The buttons 903 are located at one side of the display screen 902, and braille protrusions 904 are arranged on the upper part of the buttons 903. The working modes and states of the heating plate 4, the vibrator 6 and the heating wires 1103 are displayed through the display screen 902, the working modes and start-stop of the heating plate 4, the vibrator 6 and the heating wires 1103 are convenient to be controlled through the buttons 903. The braille protrusions 904 facilitate the blind to understand the function of the buttons 903 after touching.

A housing 901 of this embodiment is internally provided with a storage battery, a leakage protector, a controller, a vibration module, a heating module, a socket and a charging socket. The storage battery is connected with the leakage protector and the controller, the leakage protector is connected with the charging socket and the socket, and the controller is connected with the leakage protector, the vibration module, the heating module and the socket. The controller is connected with a wireless signal transmitting module, a wireless signal receiving module and a voice module. The wireless signal transmitting module, the wireless signal receiving module and the voice module are all located inside the housing 901. The leakage protector protects the use safety of gloves, the vibration module controls the working mode and start-stop of the vibrator 6, the heating module controls the working modes and start-stops of the heating plate 4 and the heating wires 1103, and the heating wires 1103 are supplied with power through connecting plug into the socket. The storage battery is charged by connecting an external charger with the charging socket. The wireless signal transmitting module and the wireless signal receiving module send the data and working modes of each device to the mobile device and receive the adjustment instructions sent by the mobile device. Voice module facilitates patients to control by voice.

The present disclosure is not limited to the above-mentioned embodiment, and anyone should know that the structural changes made under the inspiration of the present disclosure, which have the same or similar technical scheme as the present disclosure, fall within the protection scope of the present disclosure. The technologies, shapes and structural parts not described in detail in the present disclosure are all well-known technologies.

1. A glove for preventing and relieving tenosynovitis, comprising a glove body, wherein a hand warming assembly is arranged at a lower part of the glove body; a control assembly and a plurality of mounting bases are arranged at an upper part of the glove body; an insertion block is arranged on each of the mounting bases; an adsorption magnetic ring is arranged at a lower end of the insertion block; a sliding rod elastically slides at a lower part of the insertion block; a heating plate is arranged at a lower end of the sliding rod; a plurality of massage beads are arranged at a lower end of the heating plate; a vibrator is arranged at an upper end of the heating plate;
each of the mounting bases comprises a clamping sleeve, the lower part of the insertion block is slidably provided inside the clamping sleeve, and an inner wall of the clamping sleeve is circumferentially provided with a limiting magnetic ring; the adsorption magnetic ring is located at an upper end of the limiting magnetic ring; an outer wall of the clamping sleeve is circumferentially provided with a fixing plate; and the clamping sleeve and the fixing plate are both located inside the glove body.

2. The glove for preventing and relieving tenosynovitis according to claim 1, wherein the glove body sequentially comprises a fluff layer, an insulation layer, a reinforcing layer and an antifouling layer from inside to outside, a surface of the antifouling layer is formed with a circular groove corresponding to the clamping sleeve, the circular groove penetrates through the antifouling layer and the reinforcing layer, the fixing plate is located between the reinforcing layer and the insulation layer, a lower end of an inner wall of the circular groove is formed with a movable groove, the movable groove penetrates through the insulation layer, the heating plate is located in the movable groove.

3. The glove for preventing and relieving tenosynovitis according to claim 1, wherein the upper end of the limiting magnetic ring is formed with two slotted holes, and an electric-contact piece is arranged inside each of the slotted holes.

4. The glove for preventing and relieving tenosynovitis according to claim 3, wherein the adsorption magnetic ring is formed with two through holes in a vertical direction, the lower end of the insertion block is provided with a contact pin corresponding to the electric-contact piece; the contact pin penetrates corresponding one of the through holes.

5. The glove for preventing and relieving tenosynovitis according to claim 1, wherein an outer periphery of the insertion block is formed with a ring opening, the lower end of the insertion block is formed with a notch; an upper end of an inner wall of the notch is formed with a chute; an upper part of the sliding rod penetrates through the notch; an extrusion plate is installed at an upper end of the sliding rod; the extrusion plate is slidably provided inside the chute; and a spring is arranged between the extrusion plate and an upper end of an inner wall of the chute.

6. The glove for preventing and relieving tenosynovitis according to claim 1, wherein a side of the glove body is formed with an opening, an upper surface of the glove body is provided with an upper adhesive strip, and a lower surface of the glove body is provided with a lower adhesive strap corresponding to the upper adhesive strip.

7. The glove for preventing and relieving tenosynovitis according to claim 1, wherein the hand warming assembly comprise a palm layer, a hook surface is arranged on the palm layer, heating wires each arranged in an S shape are arranged inside the palm layer, and a lower surface of the glove body is provided with a loop surface corresponding to the hook surface.

8. The glove for preventing and relieving tenosynovitis according to claim 1, wherein the control assembly comprises a housing installed on an upper surface of the glove body, a display screen is installed on an upper side of the housing, a plurality of buttons are movably provided on an upper part of the housing, braille protrusions are arranged on an upper part of each of the buttons.

9. The glove for preventing and relieving tenosynovitis according to claim 8, wherein the housing is internally provided with a storage battery, a leakage protector, a controller, a vibration module, a heating module, a socket and a charging socket, the storage battery is connected with the leakage protector and the controller, the leakage protector is connected with the charging socket and the socket, and the controller is connected with the leakage protector, the vibration module, the heating module and the socket.

10. The glove for preventing and relieving tenosynovitis according to claim 9, wherein the controller is connected with a wireless signal transmitting module, a wireless signal receiving module and a voice module, and the wireless signal transmitting module, the wireless signal receiving module and the voice module are all located inside the housing.
